(19) United States
(12) Patent Application Publication
(10) Pub. No.: US 2018/0306794 A1
(43) Pub. Date: Oct. 25, 2018

(54) Methods of Producing Gene Expression Profiles of Subjects Having Cancer and Kits for Practicing Same

(71) Applicant: The Regents of the University of California, Oakland, CA (US)

(72) Inventors: Antoine Snijders, Antioch, CA (US); Jian-Hua Mao, Moraga, CA (US)

(21) Appl. No.: 15/956,602

(22) Filed: Apr. 18, 2018

Related U.S. Application Data
(60) Provisional application No. 62/487,759, filed on Apr. 20, 2017.

(51) Int. Cl. G01N 33/574; C12Q 1/6886

(52) U.S. Cl. CPC G01N 33/57415; C12Q 2600/158; C12Q 2600/106; C12Q 1/6886

(57) ABSTRACT

Provided are methods of producing gene expression profiles of subjects having cancer. In certain aspects, the methods include contacting a sample obtained from breast tumor tissue (e.g., breast tumor tissue of a Luminal A molecular subtype) with reagents for determining the expression levels of ZIC2, RGS16, SLC2A1, DDR2, PTPLAD1, CMTM8, and TROAP. In some aspects, the methods include contacting a RNA sample obtained from breast tumor tissue (e.g., breast tumor tissue of a Basal molecular subtype) with reagents for determining the expression levels of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP. Kits that find use, e.g., in practicing the methods of the present disclosure, are also provided.

### CROSS-REFERENCE TO RELATED APPLICATIONS

This application claims the benefit of U.S. Provisional Patent Application Ser. No. 62/487,759, filed Apr. 20, 2017, which application is incorporated herein by reference in its entirety.

### STATEMENT REGARDING FEDERALLY SPONSORED RESEARCH

This invention was made with Government support under contract number DE-AC02-05CH11231 awarded by The United States Department of Energy. The Government has certain rights in the invention.

### INTRODUCTION

Breast cancer (BC) is the leading female malignancy and the second leading cause of cancer deaths in U.S. women, with tumor metastasis being the underlying cause in most of these breast cancer related death. Breast carcinogenesis is a multi-step process in which epithelial cells accumulate genetic alterations, which in a permissive tissue microenvironment progress towards malignancy and may then metastasize to distant organs. Advances in imaging technologies and heightened public awareness of breast cancer have resulted in an increase in the diagnosis of early-stage breast cancer. Furthermore, adjuvant therapy has reduced the risk of recurrence and improved overall survival from BC. Radiotherapy (RT) is a well-established adjuvant treatment modality following breast cancer surgery. However, not all patients who receive radiotherapy benefit from it and could have been spared the treatment-associated side-effects including short-term effects such as skin erythema and fatigue and later side effects including telangiectasia and impaired cosmesis. Separating patients who benefit from those who do not benefit from radiotherapy remains challenging, and current clinical practice considers radiotherapy for all patients undergoing breast cancer surgery.

### SUMMARY

Provided are methods of producing gene expression profiles of subjects having cancer. In certain aspects, the methods include contacting a sample (e.g., an RNA or protein sample) obtained from breast tumor tissue (e.g., breast tumor tissue of a Luminal A molecular subtype) with reagents for determining the expression levels of ZIC2, RGS16, SLC2A1, DDR2, PTPLAD1, CMTM8, and TROAP. In some aspects, the methods include contacting a sample obtained from breast tumor tissue (e.g., breast tumor tissue of a Basal molecular subtype) with reagents for determining the expression levels of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP. Kits that find use, e.g., in practicing the methods of the present disclosure, are also provided.

### BRIEF DESCRIPTION OF THE FIGURES

FIG. 1, panels A and B, show data relating to the effect of radiotherapy on overall survival in breast cancer patients. Panel A: Kaplan-Meier overall survival curve of 1980 breast cancer patients (METABRIC) with (N=1173 patients) and without (N=807 patients) radiotherapy (p-value=0.007). Panel B: Kaplan-Meier overall survival curve of 968 breast cancer patients (TOGA) with (N=542 patients) and without (N=426 patients) radiotherapy (p-value=1.12E-04). P-values were obtained using the log rank (Mantel-Cox) test.

FIG. 2, panels A-F, show data relating to the interaction between molecular subtype and radiotherapy on overall survival in breast cancer patients from the METABRIC cohort. Kaplan-Meier overall survival curves comparing survival for breast cancer patients who received radiotherapy and those that did not receive radiotherapy across different molecular subtypes: normal like (panel A), luminal-A (panel B), luminal-B (panel C), HER2 (panel D), basal (panel E) and claudin-low (panel F). P-values were obtained using the log rank (Mantel-Cox) test.

FIG. 3, panels A-E, show data relating to the interaction between molecular subtype and radiotherapy on overall survival in breast cancer patients from the TOGA cohort. Kaplan-Meier overall survival curves comparing survival for breast cancer patients who received radiotherapy and those that did not receive radiotherapy across different molecular subtypes: normal like (panel A), luminal-A (panel B), luminal-B (panel C), HER2 (panel D) and basal (panel E). P-values were obtained using the log rank (Mantel-Cox) test.

FIG. 4, panels A-F, show data relating to the interaction between molecular subtype and radiotherapy on overall survival in breast cancer patients by meta-analysis. Kaplan-Meier overall survival curves comparing survival for breast cancer patients who received radiotherapy and those that did not receive radiotherapy across different molecular subtypes: normal like (panel A), luminal-A (panel B), luminal-B (panel C), HER2 (panel D), basal (panel E) and claudin-low (panel F). P-values were obtained using the log rank (Mantel-Cox) test.

FIG. 5, panels A-F, show data relating to the effect of radiotherapy on overall survival in younger breast cancer patients across different molecular subtypes. Kaplan-Meier overall survival curves comparing survival for breast cancer patients diagnosed at age ≤60 years (“young”) who received radiotherapy and those that did not receive radiotherapy across different molecular subtypes: normal like (panel A), luminal-A (panel B), luminal-B (panel C), HER2 (panel D), basal (panel E) and claudin-low (panel F) (METABRIC cohort). P-values were obtained using the log rank (Mantel-Cox) test.

FIG. 6, panels A-F, show data relating to the effect of radiotherapy on overall survival in older breast cancer patients across different molecular subtypes. Kaplan-Meier overall survival curves comparing survival for breast cancer patients diagnosed at age >60 years (“old”) who received radiotherapy and those that did not receive radiotherapy across different molecular subtypes: normal like (panel A), luminal-A (panel B), luminal-B (panel C), HER2 (panel D), basal (panel E) and claudin-low (panel F) (METABRIC cohort). P-values were obtained using the log rank (Mantel-Cox) test.

FIG. 7 shows data relating to gene signatures identified as predictive of survival benefit from radiotherapy in Luminal A breast cancer patients and Basal breast cancer patients.

### DETAILED DESCRIPTION

Provided are methods of producing gene expression profiles of subjects having cancer (e.g., breast cancer). In certain aspects, the methods include contacting a sample obtained from breast tumor tissue (e.g., breast tumor tissue of a Luminal A molecular subtype) with reagents for determining the expression levels of ZIC2, RGS16, SLC2A1, DDR2, PTPLAD1, CMTM8, and TROAP. In some aspects, the methods include contacting a sample obtained from breast tumor tissue (e.g., breast tumor tissue of a Basal molecular subtype) with reagents for determining the expression levels of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP. Kits that find use, e.g., in practicing the methods of the present disclosure, are also provided.

Before the methods and kits of the present disclosure are described in greater detail, it is to be understood that the methods and kits are not limited to particular embodiments described, as such may, of course, vary. It is also to be understood that the terminology used herein is for the purpose of describing particular embodiments only, and is not intended to be limiting, since the scope of the methods and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and kits. The upper and lower limits of these smaller ranges may independently be included in the smaller ranges and are also encompassed within the methods and kits, subject to any specifically excluded limit in the stated range. Where the stated range includes one or both of the limits, ranges excluding either or both of those included limits are also included in the methods and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and kits belong. Although any methods and kits similar or equivalent to those described herein can also be used in the practice or testing of the methods and kits, representative illustrative methods and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and kits, which are, for clarity, described in the context of separate embodiments, may also be provided in combination in a single embodiment. Conversely, various features of the methods and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

As summarized above, the present disclosure provides methods of producing gene expression profiles of subjects having cancer.

According to some embodiments, the methods include contacting a sample (e.g., an RNA or protein sample) obtained from breast tumor tissue from a subject having breast cancer with reagents for determining the expression levels of each of Zic family member 2 (ZIC2—NM_007129), regulator of G-protein signaling 16 (RGS16—NM_002928.3), solute carrier family 2 member 1 (SLC2A1—NM_006516.2), discoidin domain receptor tyrosine kinase 2 (DDR2—NM_001014796), protein tyrosine phosphatase-like A domain containing 1 (PTPLAD1—NM_016395), CKLF like MARVEL transmembrane domain containing 8 (CMTM8—NM_178868), and trophinin associated protein (TROAP—NM_005480), to produce a gene expression profile of the subject having breast cancer. In certain aspects, the breast tumor tissue is of a Luminal A molecular subtype. In some embodiments, the breast tumor tissue is determined to be of a Luminal A molecular subtype prior to the contacting. As demonstrated in the Experimental section below, the inventors have discovered a 7-gene signature based on expression levels of ZIC2, RGS16, SLC2A1, DDR2, PTPLAD1, CMTM8, and TROAP, that is predictive of radiotherapy benefit in Luminal A molecular subtype breast cancer patients. Accordingly, in some embodiments, the contacting step is performed only when the breast tumor tissue has been determined to be of a Luminal A molecular subtype. In certain aspects, the methods further include determining that the breast tumor tissue is of a Luminal A molecular subtype.

According to some embodiments, the methods include contacting a sample (e.g., an RNA or protein sample) obtained from breast tumor tissue from a subject having breast cancer with reagents for determining the expression levels of each of C-X-C motif chemokine ligand 13 (CXCL13—NM_006419), crystallin beta B2 (CRYBB2—NM_000496), intersectin 1 (ITSN1—NM_003024), phospholipase A1 member A (PLA1A—NM_015900), laminin subunit gamma 2 (LAMC2—NM_005562), regulator of G-protein signaling 5 (RGS5—NM_003617), WWC Family Member 3 (WWC3—NM_015691), tubulin tyrosine ligase like 7 (TTLL7—NM_024686), anaphase promoting complex subunit 1 (ANAPC1—NM_022662), tumor suppressing subtransferable candidate 1 (TSSC1—NM_001330530), complement factor H (CFH—NM_000186), HAUS augmin like complex subunit 4 (HAUS4—NM_001166269), receptor activity modifying protein 3 (RAMP3—NM_005856), mediator complex subunit 28 (MED28—NM_025205), TSC22 domain family member 3 (TSC22D3—NM_198057), LSM14A, mRNA processing body assembly factor (LSM14A—NM_001114093), and agouti signaling protein (ASIP—NM_001672), to produce a gene expression profile of the subject having breast cancer. In certain aspects, the breast tumor tissue is of a Basal molecular subtype. In some embodiments, the breast tumor tissue is determined to be of a Basal molecular subtype prior to the contacting. As demonstrated in the Experimental section below, the inventors have discovered a 17-gene signature based on expression levels of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP, that is predictive of radiotherapy benefit in Basal molecular subtype breast cancer patients. Accordingly, in some embodiments, the contacting step is performed only when the breast tumor tissue has been determined to be of a Basal molecular subtype. In certain aspects, the methods further include determining that the breast tumor tissue is of a Basal molecular subtype.

The above accession numbers beginning with “NM_” are RefSeq mRNA accession numbers. The corresponding mRNA, gene and protein sequences are known and available from a variety of nucleotide and amino acid sequence databases including the NCBI GenBank database, NCBI RefSeq database, EMBL nucleotide sequence database, and the like.

In some embodiments, the methods of the present disclosure further include producing a radiotherapy benefit score for the subject based on the determined gene expression levels. As demonstrated in the Experimental section below, the inventors have discovered that the expression levels of the specified set of genes are predictive of radiotherapy benefit in the corresponding breast cancer molecular subtype. A radiotherapy benefit score may be produced using any suitable approach based on the determined expression levels. In certain aspects, the radiotherapy benefit score is produced using a coefficient as a weighting factor for each gene. In some embodiments, such a coefficient is a proportional hazards model coefficient. For example, the coefficient may be a Cox proportional hazards model coefficient. In certain aspects, the radiotherapy benefit score is produced according to the formula: Σ (gene i β)×(gene i expression level), where the value of β for each gene is obtained from a Cox hazards regression model. Example coefficients for the various genes are provided in the Experimental section below.

In certain aspects, the methods of the present disclosure further include recommending radiotherapy to the subject to treat the breast cancer (e.g., as an adjuvant therapy) if the radiotherapy benefit score indicates that the subject would benefit from radiotherapy to treat the breast cancer. In some embodiments, radiotherapy is only recommended to an individual who is 60 years old or younger at the time of diagnosis of the breast cancer. As demonstrated in the Experimental section below, the inventors have surprisingly found that radiotherapy did not confer survival benefit in patients older than 60 years old regardless of the molecular subtype of the breast cancer. In certain aspects, when radiotherapy is recommended, the methods further include administering the radiotherapy to the subject.

In some embodiments, the methods of the present disclosure further include recommending to the subject a breast cancer treatment that does not include radiotherapy if the radiotherapy benefit score indicates that the subject would not benefit from radiotherapy to treat the breast cancer.

In certain aspects, the groups of genes independently specified above for the Luminal A and Basal molecular subtypes are the only genes of interest for which expression levels are determined. By “genes of interest” is meant genes the expression levels of which contribute to the gene expression profile (or “signature”), which do not include any genes for which expression levels are determined for purposes of controls and/or standards, e.g., housekeeping genes used as internal controls and/or standards. In other aspects, the sample may be contacted with reagents for determining the expression levels of genes of interest in addition to those of the groups of genes independently specified above for the Luminal A and Basal molecular subtypes. In certain aspects, the expression levels of 1000 or fewer, 750 or fewer, 500 or fewer, 400 or fewer, 300 or fewer, 200 or fewer, 150 or fewer, 100 or fewer, 90 or fewer, 80 or fewer, 70 or fewer, 60 or fewer, 50 or fewer, 40 or fewer, 30 or fewer, 25 or fewer, or 20 or fewer genes of interest are determined.

In some embodiments, the contacting is carried out using a panel of reagents such that gene expression levels are determined in multiplex format. For example, the contacting may include contacting a sample with gene expression detection reagents in a single container (e.g., tube, well, etc.) for determining the expression levels of two or more of the genes of interest. In some embodiments, reagents for determining the expression levels of the 7 genes specified above for the Luminal A molecular subtype or the 17 genes specified above for the Basal molecular subtype are contacted with the sample in a single container. In other embodiments, fewer than all of the reagents for determining the expression levels of the 7 genes specified above for the Luminal A molecular subtype or the 17 genes specified above for the Basal molecular subtype are contacted with the sample in a single container, and one or more separate aliquots of the sample are contacted with the remaining reagents in one or more additional containers. In certain aspects, the contacting occurs in separate containers (e.g., wells of a plate, etc.) for each gene of interest.

In certain aspects, the sample is a ribonucleic acid (RNA) sample. A wide variety of approaches may be employed to determine the gene expression levels in an RNA sample. Such approaches include, but are not limited to, quantitative reverse transcription polymerase chain reaction (qRT-PCR; also referred to as real-time RT-PCR), next-generation sequencing (NGS) (e.g., RNA-Seq), microarray analysis, Northern analysis, and the like.

In some embodiments, the sample is a protein sample. Numerous suitable approaches for determine gene expression levels in protein samples are available. Such approaches include, but are not limited to, antibody-based assays such as enzyme-linked immunosorbent assays (ELISA), flow cytometric analysis, Western analysis, and the like.

The reagents contacted with the sample will vary depending upon the type of sample (e.g., RNA versus protein) and the particular approach employed to determine the gene expression levels. For example, when the sample is an RNA sample and the approach employed is qRT-PCR, reagents suitable for reverse transcription and subsequent amplification are commercially available. Primers for amplification of cDNAs corresponding to each gene of interest are commercially available or may be designed using the available mRNA sequence information for each gene of interest. Kits and detailed guidance included therewith for performing qRT-PCR are commercially available and include, e.g., SuperScript III Platinum One-Step qRT-PCR Kit (ThermoFisher), SYBR® Green Quantitative RT-qPCR Kit (Sigma-Aldrich), etc.

As another non-limiting example, when the sample is an RNA sample and the approach employed is next-generation sequencing (NGS), the reagents for determining gene expression levels may include reagents for producing a sequencing library from the RNA sample. For example, reagents for adding sequencing adapters directly to RNAs or to cDNAs produced therefrom for quantitative NGS may be employed. By “sequencing adapter” is meant one or more nucleic acid domains that include at least a portion of a nucleic acid sequence (or complement thereof) utilized by a sequencing platform of interest, such as a sequencing platform provided by Illumina® (e.g., the HiSeq™, MiSeq™ and/or Genome Analyzer™ sequencing systems); Oxford Nanopore™ Technologies (e.g., the MinION™ sequencing system), Ion Torrent™ (e.g., the Ion PGM™ and/or Ion Proton™ sequencing systems); Pacific Biosciences (e.g., the PACBIO RS II sequencing system); Life Technologies™ (e.g., a SOLiD™ sequencing system); Roche (e.g., the 454 GS FLX+ and/or GS Junior sequencing systems); or any other sequencing platform of interest.

In certain aspects, the sequencing adapter is, or includes, a nucleic acid domain selected from: a domain (e.g., a “capture site” or “capture sequence”) that specifically binds to a surface-attached sequencing platform oligonucleotide (e.g., the P5 or P7 oligonucleotides attached to the surface of a flow cell in an Illumina® sequencing system); a sequencing primer binding domain (e.g., a domain to which the Read 1 or Read 2 primers of the Illumina® platform may bind); a unique identifier (e.g., a barcode or other domain that uniquely identifies an mRNA transcribed from a gene of interest, and/or uniquely identifies the sample source of the mRNA being sequenced to enable sample multiplexing by marking every molecule from a given sample with a specific barcode or “tag”); a barcode sequencing primer binding domain (a domain to which a primer used for sequencing a barcode binds); a molecular identification domain (e.g., a molecular index tag, such as a randomized tag of 4, 6, or other number of nucleotides) for uniquely marking molecules of interest, e.g., to determine expression levels based on the number of instances a unique tag is sequenced; a complement of any such domains; or any combination thereof.

Kits and detailed guidance included therewith for performing next-generation sequencing (e.g., RNA-seq) to determine gene expression levels from RNA samples are commercially available and include, e.g., TruSeq Stranded mRNA Library Prep Kit (Illumina), etc.

### Kits

As summarized above, the present disclosure provides kits. The kits may include, e.g., one or more of any of the reagents, buffers, etc. that find use in performing any of the methods of the present disclosure.

According to some embodiments, a kit of the present disclosure includes reagents for determining the expression levels of each of ZIC2, RGS16, SLC2A1, DDR2, PTPLAD1, CMTM8, and TROAP1, and instructions for contacting a sample obtained from breast tumor tissue from a subject having breast cancer with the reagents to produce a gene expression profile of the subject having breast cancer. In some embodiments, such instructions include instructions to perform the contacting only if the breast tumor tissue is determined to be of a Luminal A molecular subtype.

In certain aspects, a kit of the present disclosure includes reagents for determining the expression levels of each of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP, and instructions for contacting a sample obtained from breast tumor tissue from a subject having breast cancer with the reagents to produce a gene expression profile of the subject having breast cancer. In some embodiments, such instructions include instructions to perform the contacting only if the breast tumor tissue is determined to be of a Basal molecular subtype.

The kits of the present disclosure may further include reagents for determining the molecular subtype of the breast tumor tissue.

In some embodiments, the kits of the present disclosure further include instructions for producing a radiotherapy benefit score based on the gene expression profile. In certain aspects, the instructions for producing a radiotherapy benefit score are computer-readable instructions (e.g., present on a non-transitory computer-readable medium). The computer-readable medium may be physically provided with the kit (e.g., as a compact disc (CD), flash drive (e.g., a USB flash drive), or the like), or may be present on a remote server.

In some embodiments, when the kit is designed for use on RNA samples, the reagents are quantitative polymerase chain reaction (qPCR) reagents, next generation sequencing (NGS) reagents (e.g., RNA-Seq library preparation reagents, or the like), etc.

Components of the kits may be present in separate containers, or multiple components may be present in a single container. A suitable container includes a single tube (e.g., vial), one or more wells of a plate (e.g., a 96-well plate, a 384-well plate, etc.), or the like.

The instructions for contacting the sample obtained from breast tumor tissue with the reagents to produce a gene expression profile may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Computer-Readable Media and Systems

Also provided are non-transitory computer-readable media that find use in practicing embodiments of the methods of the present disclosure or one or more steps thereof. In certain aspects, provided is a non-transitory computer-readable medium that includes instructions for producing a radiotherapy benefit score based on the gene expression profiles described elsewhere herein. For example, provided is a non-transitory computer-readable medium that includes instructions for producing a radiotherapy benefit score based on the expression levels of each of ZIC2, RGS16, SLC2A1, DDR2, PTPLAD1, CMTM8, and TROAP, e.g., from breast tumor tissue of a Luminal A molecular subtype. In other aspects, provided is a non-transitory computer-readable medium that includes instructions for producing a radiotherapy benefit score based on the expression levels of each of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP, e.g., from breast tumor tissue of a Basal molecular subtype.

Systems including any of the non-transitory computer-readable media of the present disclosure are also provided. Such systems may include, e.g., a processor and the non-transitory computer-readable medium, where the non-transitory computer-readable medium includes instructions that cause the processor to producing a radiotherapy benefit score based on the expression levels of the sets of genes set forth above, that is—each of ZIC2, RGS16, SLC2A1, DDR2, PTPLAD1, CMTM8, and TROAP, e.g., from breast tumor tissue of a Luminal A molecular subtype; or each of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP, e.g., from breast tumor tissue of a Basal molecular subtype.

The following examples are offered by way of illustration and not by way of limitation.

### EXPERIMENTAL

### Introduction

Breast cancer (BC) is the most common cancer in females worldwide and the second leading cause of cancer death in U.S. women. Adjuvant radiotherapy (RT) is often used to eradicate remaining tumor cells following surgery with the goal of increasing overall survival. Despite its widespread use, RT does not provide a survival benefit for all patients.

The current study investigated the impact of age and breast cancer molecular subtype on overall survival after RT. The METABRIC and TCGA breast cancer patient cohorts, which contain 1980 and 1100 breast cancer samples, respectively, were stratified by age and molecular subtype to investigate survival benefit associated with RT using Kaplan-Meier analysis. A survival benefit of RT for the luminal-A and basal breast cancer molecular subtypes was observed. Stratifying patients based on age revealed that the survival benefit is restricted to younger patients (≤60 years of age at diagnosis) whereas RT in older breast cancer patients (>60 years of age at diagnosis) offers little survival benefit. The survival benefit associated with RT was independent of clinical factors including tumor size, estrogen and progesterone status, tumor grade and molecular subtype. There is a significant survival benefit of radiotherapy for younger patients with tumors of the luminal A and basal molecular subtype. Patients with other breast tumor subtypes or older breast cancer patients could be spared the harmful effects of RT. Additional treatment strategies should be considered for these patients.

### Methods

Patient Cohorts

Clinical data for TCGA and METABRIC cohorts were obtained from cBioPortal for 1100 breast cancer samples (TCGA) and for 1980 breast cancer samples (METABRIC). Additional clinical data including PAM50, patient age at diagnosis and radiotherapy for the TCGA cohort was obtained from the UCSC Genome Browser and UCSC Cancer Browser. Summary details of the two patient cohorts are presented in Table 1.

Survival and Statistical Analysis

All survival and statistical analyses were performed using SPSS. Kaplan-Meier survival curves were generated to show differences in overall survival (p-values were generated using log rank (Mantel-Cox) test) between patients with and without radiotherapy. Significance level was set at p<0.05.

### Example 1—Radiotherapy Improves Overall Survival in Breast Cancer Patients

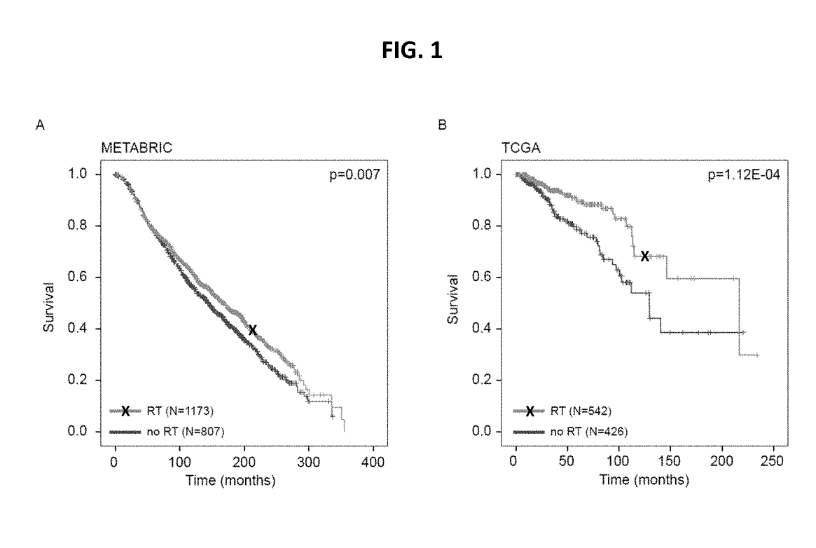

Evidence has shown that radiotherapy after BC surgery leads to increased patient survival. To further validate this observation, two large BC patient cohorts (METABRIC and TCGA) were used, which contain clinical data including radiotherapy, molecular subtype, age, overall survival (OS) and other patient characteristics. The demographic differences between patients that received radiotherapy versus those that did not were first investigated using the METABRIC data (Table 1). A higher proportion of young patients, and patients with high grade and late stage tumors received radiotherapy (Table 1). Overall we found that patients who receive radiotherapy survive significantly longer compared to those who did not receive radiotherapy in both datasets (FIG. 1; METABRIC: p-value=0.007; TCGA: p-value=1.12E-04).

TABLE 1
Distribution of clinical characteristics
of METABRIC breast cancer cohorts.

| | Radiotherapy | | |
|---|---|---|---|
| | no | yes | p-value |
| Age (mean +/− st. dev) | 63.1 (12.8) | 59.7 (12.9) | 1.05E−08 |
| Age ≤ 60 years* | 303 | 572 | |
| Age > 60 years* | 500 | 599 | |
| Tumor size (mean +/− st. dev) | 25.8 (13.3) | 26.5 (16.7) | 0.098 |
| ER status* | | | 0.292 |
| Neg | 158 | 316 | |
| Pos | 649 | 857 | |
| PR status* | | | 0.015 |
| Neg | 359 | 581 | |
| Pos | 448 | 592 | |
| Histological Grade* | | | 1.42E−09 |
| I | 85 | 84 | |
| II | 353 | 418 | |
| III | 309 | 643 | |
| Chemotherapy* | | | 2.07E−14 |
| No | 730 | 838 | |
| Yes | 77 | 335 | |
| Hormone therapy* | | | 9.85E−06 |
| No | 359 | 405 | |
| Yes | 448 | 768 | |
| Stage* | | | 4.91E−09 |
| 0 | 9 | 3 | |
| 1 | 212 | 289 | |
| 2 | 265 | 560 | |
| 3 | 18 | 100 | |
| 4 | 5 | 5 | |
| PAM50 subtype | | | 6.91E−05 |
| Normal-like | 74 | 74 | |
| Luminal A | 322 | 378 | |
| Luminal B | 176 | 299 | |
| Her2 | 68 | 141 | |
| Basal | 73 | 145 | |
| Claudin-low | 803 | 1171 | |

*Number of patients

### Example 2—Impact of Radiotherapy on Patient Survival is Independent of Clinical Factors

To determine if the impact of radiotherapy on patient survival was independent of age at diagnosis, tumor size, chemotherapy, hormone therapy, estrogen- and progesterone-receptor status, tumor grade and molecular subtype (as determined by Pam50), multivariate Cox regression with these factors including radiotherapy as covariates was used. In multivariate analysis the difference of OS attributable to radiotherapy remained significant (HR=0.811: 95% CI: 0.714-0.922; p=1.39E-03). Also found was that age, tumor size, chemotherapy, ER status, tumor grade, PAM50 subtype were significantly associated with OS (Table 2). Surprisingly, the hazard ratio for chemotherapy was 1.851 (95% CI:1.522-2.251; p=7.1E-10) suggesting that chemotherapy does not confer a survival benefit in this population based study.

TABLE 2
Prognosis Factors in Multivariate Analyses.

| | | Hazard | | |
|---|---|---|---|---|
| | | Ratio | 95% CI for HR | |
| Factors | p-value | (HR) | Lower | Upper |
| Radiotherapy | 1.389E−03 | .811 | .714 | .922 |
| Age (years) | 2.387E−42 | 1.043 | 1.037 | 1.049 |
| Tumor size (mm) | 6.934E−12 | 1.011 | 1.008 | 1.015 |
| Chemotherapy | 7.113E−10 | 1.851 | 1.522 | 2.251 |
| Hormone therapy | 4.514E−01 | 1.055 | .918 | 1.213 |
| ER status | 2.186E−02 | .753 | .591 | .960 |
| PR status | 1.827E−01 | .905 | .780 | 1.048 |
| Grade | 9.087E−03 | | | |
| Grade II vs I | 1.627E−01 | 1.194 | .931 | 1.532 |
| Grade III vs I | 8.311E−03 | 1.416 | 1.094 | 1.834 |
| Pam50 subtype | 2.852E−04 | | | |
| Luminal A vs Normal-like | 3.651E−02 | .763 | .593 | .983 |
| Luminal B vs Normal-like | 5.803E−01 | .929 | .714 | 1.207 |
| Her2 vs Normal-like | 4.094E−01 | .881 | .652 | 1.190 |
| Basal vs Normal-like | 1.368E−02 | .651 | .463 | .916 |
| Claudin-low vs Normal-like | 4.509E−04 | .547 | .391 | .766 |

### Example 3—Molecular Subtype-Specific Impact of Radiotherapy on Patient Survival

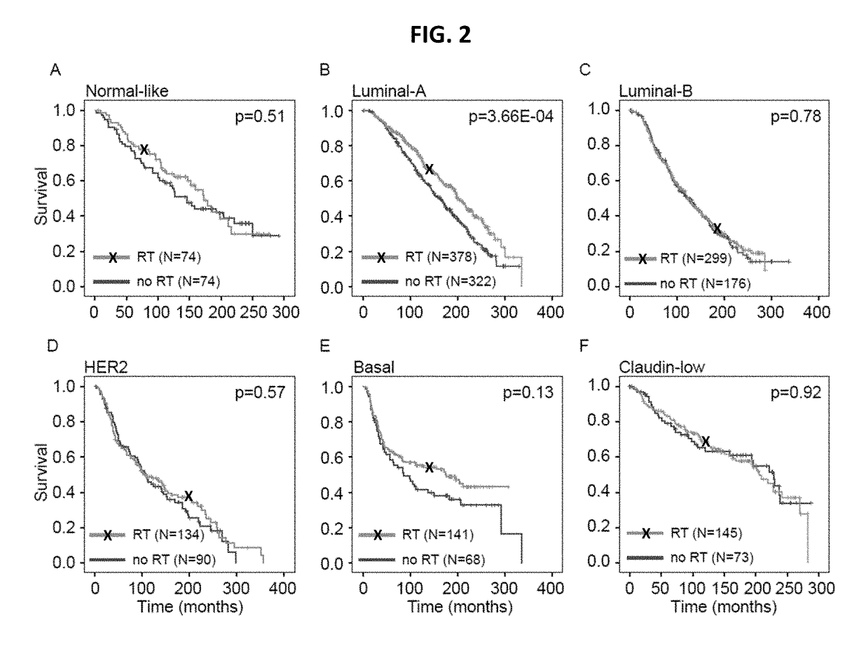

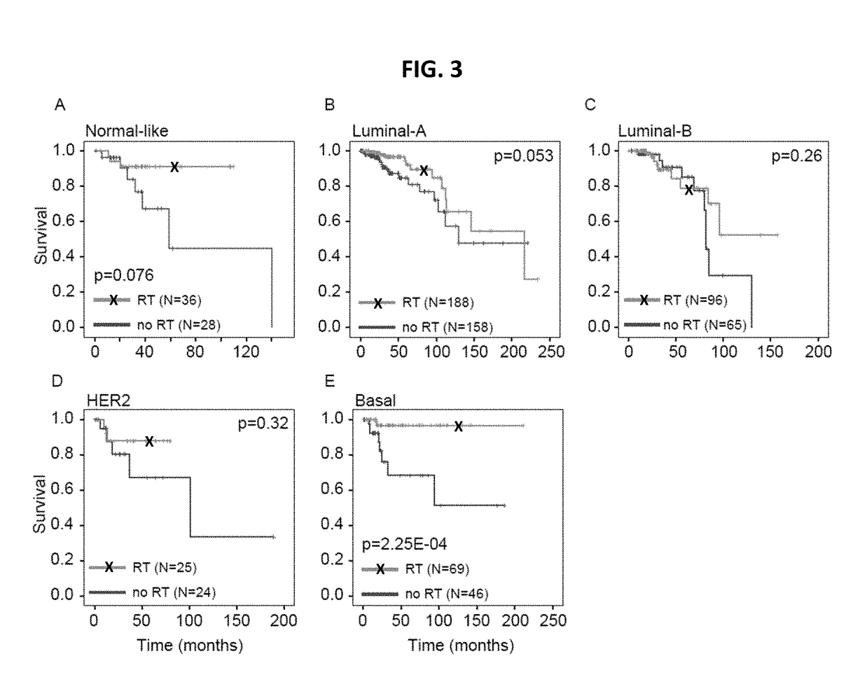

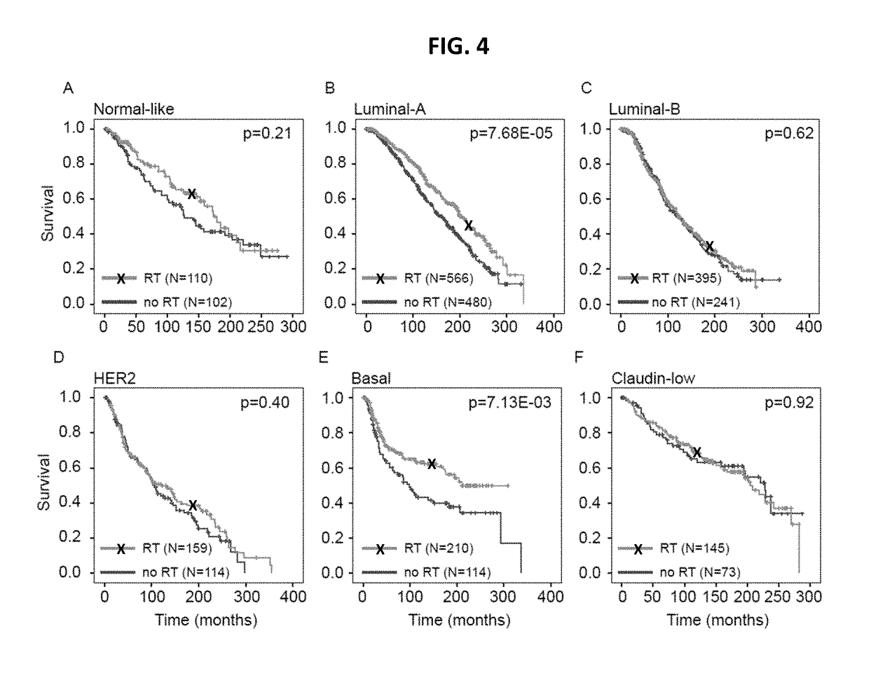

BC is a heterogeneous disease and gene expression signatures have been developed that classify breast tumors into six different molecular subtypes (normal-like, luminal A, luminal B, HER2, basal and claudin-low). See Perou et al. (2000) Nature 406:747-752; Prat & Perou (2011) Molecular Oncology 5:5-23; Prat et al. (2012) Breast Cancer Research and Treatment 135:301-306; and Prat et al. (2010) Breast Cancer Research 12:R68. Studies have demonstrated an association between molecular subtype and patient prognosis. The basal and HER2 subtypes are generally more aggressive and associated with poorer survival compared to normal-like and luminal breast tumors. To investigate whether radiotherapy benefits patients equally among different molecular subtypes, the patient cohorts were stratified into different molecular subtypes based on the PAM50 molecular score (see Parker et al. (2009) Journal of Clinical Oncology 27:1160-1167). Surprisingly, in the METABRIC cohort, it was found that radiotherapy increased patient survival only in the luminal A subtype (p=3.66E-04), whereas a tendency for increased survival was observed for the basal subtypes (p=0.13) (FIG. 2). No survival benefit was observed for the other subtypes (normal-like: p=0.51; luminal B: p=0.78; HER2: p=0.57; and claudin-low: p=0.92) (FIG. 2). In the TOGA cohort, radiotherapy significantly increased overall survival only in the basal subtype (p=2.25E-04) (FIG. 3). A tendency for survival benefit associated with radiotherapy was observed for the normal-like (p=0.076) and luminal A (p=0.053) subtypes (FIG. 3). Again, no survival benefit was observed for the luminal B (p=0.26) and HER2 (p=0.32) subtypes (FIG. 3). A meta-analysis combining the METABRIC and TOGA cohorts confirmed that radiotherapy significantly increased patient survival in the luminal A (p=7.68E-05) and basal subtypes (p=7.13E-03) (FIG. 4). Accordingly, it was concluded that there is a likely survival benefit associated with radiotherapy in the luminal A and basal subtypes.

### Example 4—Effect of Age at Diagnosis on Patient Survival after Radiotherapy

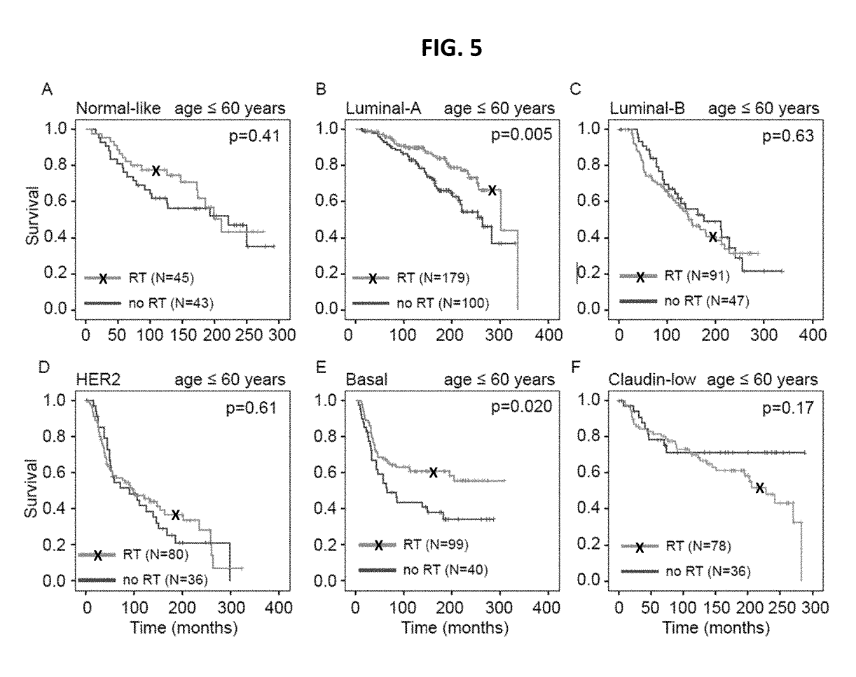

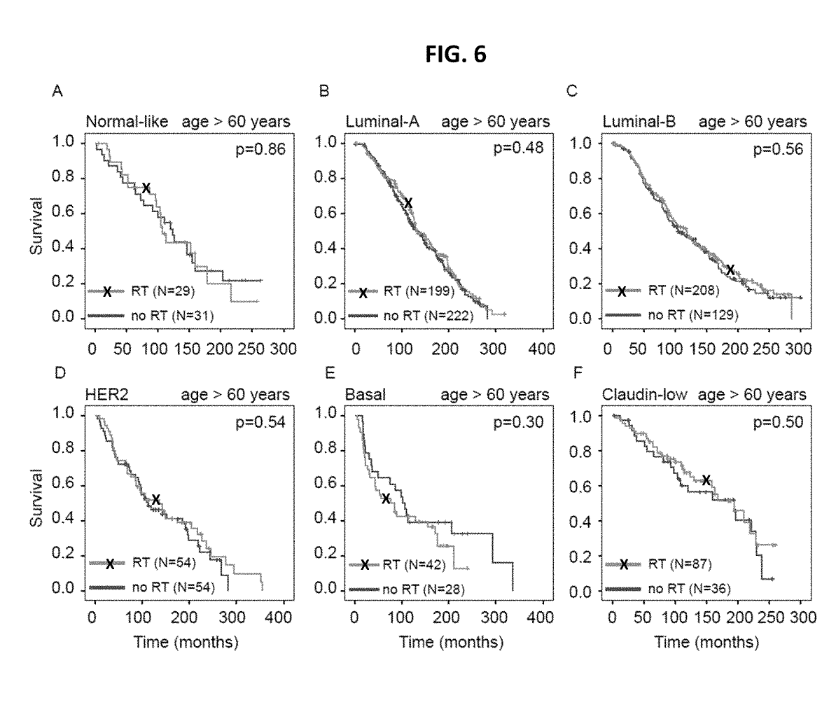

Age at diagnosis is another known prognostic factor in BC. Investigated was whether age at diagnosis in combination with molecular subtype could further clarify the survival benefit from radiotherapy. The patient cohorts were split into two age groups: “young” (age ≤60 years) and “old” (age >60 years). In the “young” group, radiotherapy significantly increased overall survival in luminal A (p=0.005) and basal (p=0.020) subtypes (FIG. 5). No survival benefit was observed in other subtypes (FIG. 5; normal-like: p=0.41; luminal B: p=0.63; HER2: p=0.61 and claudin-low: p=0.17). Surprisingly, in the “old” group, radiotherapy did not confer survival benefit for any molecular subtype (FIG. 6; normal-like: p=0.86; luminal A: p=0.48; luminal B: p=0.56; HER2: p=0.54, basal: p=0.30 and claudin-low: p=0.50). Accordingly, it was concluded that age at initial diagnosis significantly influences the survival associated with radiotherapy in specific molecular subtypes.

### Example 5—Identification of Gene Signatures that Predict Survival Benefit from Radiotherapy in Luminal A and Basal Molecular Subtype Breast Cancer Patients

A genome-wide screen was performed to identify genes significantly differentially expressed between patients that survive >10 years after radiotherapy versus those that survive <10 years after radiotherapy. Identified were 57 genes in Luminal A molecular subtype breast cancers and 41 genes in Basal molecular subtype breast cancers.

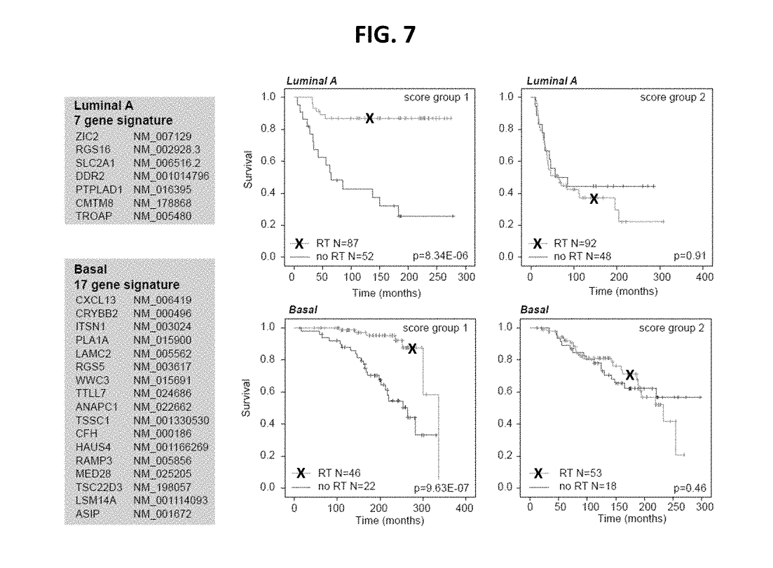

A resampling together with a conditional Cox regression analysis was used to identify a 7 gene signature in Luminal A and a 17 gene signature in Basal type breast cancers, which predict survival benefit from radiotherapy (FIG. 7).

For the Luminal A molecular subtype, the gene signature involves expression levels of ZIC2, RGS16, SLC2A1, DDR2, PTPLAD1, CMTM8, and TROAP.

For the Basal molecular subtype, the gene signature involves expression levels of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP.

### Example 6—Radiotherapy Benefit Scoring System Based on Luminal A and Basal Gene Signatures

A radiotherapy benefit scoring system was created based on the gene signatures described in the preceding example for the corresponding subtypes. A prognostic score may then be calculated, e.g., based on the following formula:

Σ(gene iβ)×(gene i expression level)

The value of β for each gene is a weighting factor for each gene obtained from a Cox proportional hazards regression model. The coefficient may change slightly depending on the platform that is used to assess gene expression levels. Example coefficients for Luminal A and Basal molecular subtypes are shown in Tables 3 and 4, respectively.

TABLE 3
Example coefficients for Luminal A gene signature
Luminal A

| Gene Name | Coefficient |
|---|---|
| ZIC2 | .793 |
| RGS16 | 1.919 |
| SLC2A1 | 1.184 |
| DDR2 | .978 |
| PTPLAD1 | 1.010 |
| CMTM8 | .737 |
| TROAP | 1.385 |

TABLE 4
Example coefficients for Basal gene signature
Basal

| Gene Name | Coefficient |
|---|---|
| CXCL13 | −1.641 |
| CRYBB2P1 | −1.865 |
| ITSN1 | 1.654 |
| PLA1A | 2.045 |
| LAMC2 | −.455 |
| RGS5 | −2.026 |
| WWC3 | −.989 |
| TTLL7 | −1.865 |
| ANAPC1 | 1.881 |
| TSSC1 | −1.703 |
| CFH | −1.578 |
| HAUS4 | −1.964 |
| RAMP3 | 1.769 |
| MED28 | 1.394 |
| TSC22D3 | 1.166 |
| LSM14A | −.864 |
| ASIP | −2.165 |

### Example 7—Radiotherapy Recommendation in Luminal A or Basal Breast Cancer Patients Based on Gene Signatures/Scoring System

The above-described signatures/scoring system may be used to guide clinicians as to whether or not radiotherapy after surgery will be beneficial in patients with Luminal A or Basal breast cancer. An example embodiment is described below.

After surgery, RNA is extracted from the tumor tissue and the expression levels of the genes set forth in Example 5 are measured using a method such as Taqman, Panomic, Nanostring, RNAseq, etc. Based on the score, the patient is assigned to score group A, score group B, or score group C. Radiotherapy treatment would be recommended to patients in score group A. Radiotherapy treatment would not be recommended to patients in score group C. Patients in score group B would be advised that radiotherapy may be beneficial to treat the breast cancer.

Notwithstanding the appended claims, the disclosure is also defined by the following clauses:

1. A method of producing a gene expression profile of a subject having breast cancer, comprising:
contacting a sample obtained from breast tumor tissue from a subject having breast cancer with reagents for determining the expression levels of each of ZIC2, RGS16, SLC2A1, DDR2, PTPLAD1, CMTM8, and TROAP,
to produce a gene expression profile of the subject having breast cancer.
   2. The method according to Clause 1, wherein the breast tumor tissue is of a Luminal A molecular subtype.
   3. The method according to Clause 2, wherein the breast tumor tissue is determined to be of a Luminal A molecular subtype prior to the contacting.
   4. The method according to Clause 3, wherein the contacting is performed only when the breast tumor tissue has been determined to be of a Luminal A molecular subtype.
   5. The method according to any one of Clauses 2 to 4, further comprising determining that the breast tumor tissue is of a Luminal A molecular subtype.
   6. A method of producing a gene expression profile of a subject having breast cancer, comprising:
contacting a sample obtained from breast tumor tissue from a subject having breast cancer with reagents for determining the expression levels of each of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP,
to produce a gene expression profile of the subject having breast cancer.
   7. The method according to Clause 6, wherein the breast tumor tissue is of a Basal molecular subtype.
   8. The method according to Clause 7, wherein the breast tumor tissue is determined to be of a Basal molecular subtype prior to the contacting.
   9. The method according to Clause 8, wherein the contacting is performed only when the breast tumor tissue has been determined to be of a Basal molecular subtype.
   10. The method according to any one of Clauses 7 to 9, further comprising determining that the breast tumor tissue is of a Basal molecular subtype.
   11. The method according to any one of Clauses 1 to 10, further comprising producing a radiotherapy benefit score for the subject based on the determined expression levels.
   12. The method according to Clause 11, wherein the radiotherapy benefit score is produced using a coefficient as a weighting factor for each gene.
   13. The method according to Clause 12, wherein the coefficient is a proportional hazards model coefficient.
   14. The method according to Clause 13, wherein the coefficient is a Cox proportional hazards model coefficient.
   15. The method according to Clause 14, wherein the radiotherapy benefit score is produced according to the following formula:

Σ(gene iβ)×(gene i expression level),
wherein the value of β for each gene is obtained from a Cox hazards regression model.
   16. The method according to any one of Clauses 11 to 15, further comprising recommending radiotherapy to the subject to treat the breast cancer if the radiotherapy benefit score indicates that the subject would benefit from radiotherapy to treat the breast cancer.
   17. The method according to Clause 16, wherein the radiotherapy is only recommended to an individual who is 60 years old or younger at the time of diagnosis of the breast cancer.
   18. The method according to Clause 16 or Clause 17, further comprising administering the radiotherapy to the subject.
   19. The method according to any one of Clauses 11 to 15, further comprising recommending to the subject a breast cancer treatment that does not include radiotherapy if the radiotherapy benefit score indicates that the subject would not benefit from radiotherapy to treat the breast cancer.
   20. The method according to any one of Clauses 1 to 19, wherein the expression levels of 100 or fewer genes of interest are determined.
   21. The method according to any one of Clauses 1 to 19, wherein the expression levels of 50 or fewer genes of interest are determined.
   22. The method according to any one of Clauses 1 to 19, wherein the expression levels of 25 or fewer genes of interest are determined.
   23. The method according to any one of Clauses 1 to 19, wherein the expression levels of no more than 25 genes are determined.
   24. The method according to any one of Clauses 1 to 23, wherein the sample is a ribonucleic acid (RNA) sample.
   25. The method according to Clause 24, wherein the expression levels are determined by quantitative polymerase chain reaction (qPCR).
   26. The method according to Clause 24, wherein the expression levels are determined by next generation sequencing (NGS).
   27. The method according to any one of Clauses 1 to 23, wherein the sample is a protein sample.
   28. The method according to Clause 27, wherein the expression levels are determined using an antibody-based assay.
   29. The method according to Clause 28, wherein the antibody-based assay is an enzyme-linked immunosorbent assay (ELISA).
   30. A kit, comprising:
reagents for determining the expression levels of each of ZIC2, RGS16, SLC2A1, DDR2, PTPLAD1, CMTM8, and TROAP; and
instructions for contacting a sample obtained from breast tumor tissue from a subject having breast cancer with the reagents to produce a gene expression profile of the subject having breast cancer.
   31. The kit of Clause 30, wherein the instructions comprise instructions to perform the contacting only if the breast tumor tissue is determined to be of a Luminal A molecular subtype.
   32. The kit of Clause 30 or Clause 31, wherein the kit comprises reagents for determining the expression levels of no more than 20 genes.
   33. The kit of Clause 30 or Clause 31, wherein the kit comprises reagents for determining the expression levels of no more than 15 genes.
   34. The kit of Clause 30 or Clause 31, wherein the kit comprises reagents for determining the expression levels of no more than 10 genes.
   35. A kit, comprising:
reagents for determining the expression levels of each of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP; and
instructions for contacting a sample obtained from breast tumor tissue from a subject having breast cancer with the reagents to produce a gene expression profile of the subject having breast cancer.
   36. The kit of Clause 35, wherein the instructions comprise instructions to perform the contacting only if the breast tumor tissue is determined to be of a Basal molecular subtype.
   37. The kit of Clause 35 or Clause 36, wherein the kit comprises reagents for determining the expression levels of no more than 30 genes.
   38. The kit of Clause 35 or Clause 36, wherein the kit comprises reagents for determining the expression levels of no more than 25 genes.
   39. The kit of Clause 35 or Clause 36, wherein the kit comprises reagents for determining the expression levels of no more than 20 genes.
   40. The kit of any one of Clauses 30 to 39, further comprising reagents for determining the molecular subtype of the breast tumor tissue.
   41. The kit of any one of Clauses 30 to 40, further comprising instructions for producing a radiotherapy benefit score based on the gene expression profile.
   42. The kit of Clause 41, wherein the instructions for producing a radiotherapy benefit score are computer-readable instructions.
   43. The kit of any one of Clauses 30 to 42, wherein the reagents are quantitative polymerase chain reaction (qPCR) reagents.
   44. The kit of any one of Clauses 30 to 42, wherein the reagents are next generation sequencing (NGS) reagents.
   45. A non-transitory computer-readable medium comprising instructions for producing a radiotherapy benefit score.
   46. The non-transitory computer-readable medium of Clause 45, comprising instructions for producing a radiotherapy benefit score based on the expression levels of each of ZIC2, RGS16, SLC2A1, DDR2, PTPLAD1, CMTM8, and TROAP.
   47. The non-transitory computer-readable medium of Clause 46, wherein the expression levels are from a sample obtained from breast tumor tissue of a Luminal A molecular subtype.
   48. The non-transitory computer-readable medium of Clause 45, comprising instructions for producing a radiotherapy benefit score based on the expression levels of each of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP.
   49. The non-transitory computer-readable medium of Clause 48, wherein the expression levels are from a sample obtained from breast tumor tissue of a Basal molecular subtype.
   50. A system, comprising:
a processor; and
the non-transitory computer-readable medium of any one of Clauses 45-49, wherein the non-transitory computer-readable medium causes the processor to produce a radiotherapy benefit score.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A method of producing a gene expression profile of a subject having breast cancer, comprising:
contacting a sample obtained from breast tumor tissue from a subject having breast cancer with reagents for determining the expression levels of each of ZIC2, RGS16, SLC2A1, DDR2, PTPLAD1, CMTM8, and TROAP,
to produce a gene expression profile of the subject having breast cancer.

2. The method according to claim 1, wherein the breast tumor tissue is of a Luminal A molecular subtype.

3. The method according to claim 2, wherein the breast tumor tissue is determined to be of a Luminal A molecular subtype prior to the contacting.

4. The method according to claim 3, wherein the contacting is performed only when the breast tumor tissue has been determined to be of a Luminal A molecular subtype.

5. The method according to claim 2, further comprising determining that the breast tumor tissue is of a Luminal A molecular subtype.

6. A method of producing a gene expression profile of a subject having breast cancer, comprising:
contacting a sample obtained from breast tumor tissue from a subject having breast cancer with reagents for determining the expression levels of each of CXCL13, CRYBB2, ITSN1, PLA1A, LAMC2, RGS5, WWC3, TTLL7, ANAPC1, TSSC1, CFH, HAUS4, RAMP3, MED28, TSC22D3, LSM14A, and ASIP,
to produce a gene expression profile of the subject having breast cancer.

7. (canceled)

8. The method according to claim 6, wherein the breast tumor tissue is determined to be of a Basal molecular subtype prior to the contacting.

9. The method according to claim 8, wherein the contacting is performed only when the breast tumor tissue has been determined to be of a Basal molecular subtype.

10. (canceled)

11. The method according to claim 1, further comprising producing a radiotherapy benefit score for the subject based on the determined expression levels.

12. The method according to claim 11, wherein the radiotherapy benefit score is produced using a coefficient as a weighting factor for each gene.

13. The method according to claim 12, wherein the coefficient is a proportional hazards model coefficient.

14.-15. (canceled)

16. The method according to claim 11, further comprising recommending radiotherapy to the subject to treat the breast cancer if the radiotherapy benefit score indicates that the subject would benefit from radiotherapy to treat the breast cancer.

17. The method according to claim 16, wherein the radiotherapy is only recommended to an individual who is 60 years old or younger at the time of diagnosis of the breast cancer.

18. The method according to claim 16, further comprising administering the radiotherapy to the subject.

19. The method according to claim 11, further comprising recommending to the subject a breast cancer treatment that does not include radiotherapy if the radiotherapy benefit score indicates that the subject would not benefit from radiotherapy to treat the breast cancer.

20. The method according to claim 1, wherein the expression levels of 100 or fewer genes of interest are determined.

21.-23. (canceled)

24. The method according to claim 1, wherein the sample is a ribonucleic acid (RNA) sample.

25. The method according to claim 24, wherein the expression levels are determined by quantitative polymerase chain reaction (qPCR), next generation sequencing (NGS), or both.

26. (canceled)

27. The method according to claim 1, wherein the sample is a protein sample.

28. The method according to claim 27, wherein the expression levels are determined using an antibody-based assay.

29.-50. (canceled)
